(19) United States
(12) Patent Application Publication
(10) Pub. No.: US 2021/0188866 A1
(43) Pub. Date: Jun. 24, 2021

(54) METHOD FOR PREPARING HIGH-PURITY TOTAL GINKGOLIDE AND USE THEREOF

(71) Applicant: SPH Xing Ling Sci. & Tech. Pharmaceutical Co., Ltd., Shanghai (CN)

(72) Inventors: Shuang LIANG, Shanghai (CN); Xiangwei ZHENG, Shanghai (CN); Yue DU, Shanghai (CN); Qi GAO, Shanghai (CN); Dandan WANG, Shanghai (CN); Guoqin ZHU, Shanghai (CN)

(73) Assignee: SPH Xing Ling Sci. & Tech. Pharmaceutical Co., Ltd., Shanghai (CN)

(21) Appl. No.: 16/757,759

(22) PCT Filed: Apr. 10, 2018
(86) PCT No.: PCT/CN2018/082545

§ 371 Date: Apr. 20, 2020

(30) Foreign Application Priority Data
Jan. 19, 2018 (CN) 201810051385.2

(51) Int. Cl. C07D 493/22

(52) U.S. Cl. CPC C07D 493/22

(57) ABSTRACT

The present disclosure relates to the field of pharmacy, specifically to a method for preparing a high-purity ginkgolide and a use thereof. The method for preparing total ginkgolide includes the following steps: (1) mixing a Ginkgo biloba extract powder with an alkali solution, obtaining a precipitate by dissolving and centrifuging; (2) obtaining a total ginkgolide concentrate through purifying the precipitate by silica gel column and recrystallizing. The preparation method of total ginkgolide provided in the present disclosure can improve the purity of total ginkgolide, and the purity of the prepared total ginkgolide is ≥80%, which has good clinical application prospect; and the preparation process does not require complicated multi-step recrystallization step, and the total ginkgolide yield is high.

### CROSS REFERENCES TO RELATED APPLICATIONS

This is a Sect. 371 National Stage application of a PCT International Application No. PCT/CN2018/082545, filed on Apr. 10, 2018, which claims priority of a Chinese Patent Application No. 2018100513852, filed on Jan. 19, 2018, the contents of both applications are hereby incorporated by reference in their entireties for all purposes.

### BACKGROUND OF THE PRESENT DISCLOSURE

### Field of Disclosure

The present disclosure relates to the technical field of pharmacy, in particular, to a method for preparing a high-purity total ginkgolide and use thereof.

### Description of Related Arts

Ginkgo biloba extract powder (GBE50) is a kind of product with active ingredients extracted from the leaves of Ginkgo biloba L. by using appropriate solvents. Preparations made of GBE50 are widely used in medicine, health care products, food additives, functional beverages, cosmetics and other fields. Among them, in the field of medicine, Ginkgo biloba extract can be used for chest stuffiness and pains, stroke, hemiplegia, stiff tongue and sluggish speech caused by embolism stasis blocking channels; coronary heart disease stable angina pectoris, cerebral infarction, etc.

The total ginkgolide compound belongs to the terpenoid compound and includes sesquiterpene lactone and diterpene lactone. It is an important active ingredient in Ginkgo leaves.

The purity of the total ginkgolide prepared by conventional means is only 6%, which limits the application and therapeutic effect of the total ginkgolide. How to prepare high-purity total ginkgolide has been a research hotspot in the field of pharmacy.

### SUMMARY OF THE PRESENT DISCLOSURE

The object of the present disclosure is to provide a method for preparing a high-purity total ginkgolide and use thereof for preparing high-purity total ginkgolide having a purity of ≥80%.

In order to accomplish the above and other related objects, a first aspect of the present disclosure provides a method for preparing total ginkgolide, comprising the steps of: (1) mixing a Ginkgo biloba extract powder with an alkali solution, obtaining a precipitate by dissolving and centrifuging e; (2) obtaining a total ginkgolide concentrate through purifying the precipitate by a silica gel column and recrystallizing.

In one embodiment of the present disclosure, the filler in the silica gel column is silica gel.

In one embodiment of the present disclosure, the filler in the silica gel column is pretreated with chloroform:methanol=10:1 to 100:1.

In one embodiment of the present disclosure, the filler in the silica gel column is pretreated with chloroform:methanol=10:1 to 50:1.

In one embodiment of the present disclosure, the filler in the silica gel column is pretreated with chloroform:methanol=10:1 to 40:1.

In one embodiment of the present disclosure, the filler in the silica gel column is pretreated with chloroform:methanol=10:1 to 30:1.

In one embodiment of the present disclosure, the filler in the silica gel column is pretreated with chloroform:methanol=30:1 to 100:1.

In one embodiment of the present disclosure, the filler in the silica gel column is pretreated with chloroform:methanol=30:1 to 50:1.

In one embodiment of the present disclosure, the filler in the silica gel column is pretreated with chloroform:methanol=30:1 to 40:1.

In one embodiment of the present disclosure, the filler in the silica gel column is pretreated with chloroform:methanol=40:1 to 100:1.

In one embodiment of the present disclosure, the filler in the silica gel column is pretreated with chloroform:methanol=40:1 to 50:1.

In one embodiment of the present disclosure, the filler in the silica gel column is pretreated with chloroform:methanol=50:1 to 100:1.

It should be noted that the ratio between chloroform and methanol is a volume ratio, and in the embodiments, the ratio between chloroform and methanol is also a volume ratio.

Further, the volume ratio of chloroform:methanol may also be 10:1, 30:1, 40:1, 50:1, and 100:1.

In one embodiment of the present disclosure, the filler in the silica gel column has a mesh number of 100 to 300.

In one embodiment of the present disclosure, the filler in the silica gel column has a mesh number of 200 to 300.

In one embodiment of the present disclosure, the filler in the silica gel column has a mesh number of 100 to 200.

In one embodiment of the present disclosure, the silica gel column has a diameter to height ratio of 1:4 to 1:10.

In one embodiment of the present disclosure, the silica gel column has a diameter to height ratio of 1:4 to 1:7.

In one embodiment of the present disclosure, the silica gel column has a diameter to height ratio of 1:7 to 1:10.

In one embodiment of the present disclosure, the silica gel column has a diameter to height ratio of 1:7, 1:4, or 1:10.

In one embodiment of the present disclosure, the absorption ratio of the silica gel column is 1:30 to 1:50.

In one embodiment of the present disclosure, the absorption ratio of the silica gel column is 1:30 to 1:40.

In one embodiment of the present disclosure, the absorption ratio of the silica gel column is 1:40 to 1:50.

In one embodiment of the present disclosure, the absorption ratio of the silica gel column may be 1:40, 1:30 or 1:50.

The absorption ratio refers to the mass ratio of the loaded sample and the used resin.

In one embodiment of the present disclosure, the step (2) comprises: dissolving the precipitate in dichloromethane:methanol=1:1 to 3:1, and loading the silica gel column.

It should be noted that the ratio between dichloromethane and methanol is a volume ratio, and in the embodiments, the ratio between dichloromethane and methanol is a volume ratio.

In one embodiment of the present disclosure, the step (2) comprises: dissolving the precipitate in dichloromethane:methanol=1:1 or 3:1, and loading the silica gel column.

In one embodiment of the present disclosure, after loading the silica gel column, a first elution is carried out with dichloromethane:methanol=42:1 to 38:1 to obtain a first eluate.

It should be noted that in the present embodiment, the ratio between dichloromethane and methanol is a volume ratio.

In one embodiment of the present disclosure, the first elution is carried out with dichloromethane:methanol=40:1 to 42:1.

In one embodiment of the present disclosure, the first elution is carried out with dichloromethane:methanol=40:1 to 38:1.

In one embodiment of the present disclosure, the first elution is carried out with dichloromethane:methanol=40:1, 42:1 or 38:1.

In one embodiment of the present disclosure, after the first elution, a second elution is carried out with dichloromethane:methanol=37:1 to 33:1 to obtain a second eluate.

It should be noted that the ratio between dichloromethane and methanol is a volume ratio, and in the embodiments, the ratio between dichloromethane and methanol is a volume ratio.

In one embodiment of the present disclosure, the second elution is carried out with dichloromethane:methanol=37:1 to 35:1.

In one embodiment of the present disclosure, the second elution is carried out with dichloromethane:methanol=33:1 to 35:1.

In one embodiment of the present disclosure, the second elution is carried out with dichloromethane:methanol=35:1, 37:1 or 33:1.

In one embodiment of the present disclosure, the step (2) comprises: dissolving the precipitate in dichloromethane:methanol=1:1 or 3:1, loading the silica gel column, carrying out the first elution with dichloromethane:methanol=40:1 to obtain the first eluate, and carrying out the second elution with dichloromethane:methanol=35:1 to obtain the second eluate; respectively drying and recrystallizing the first eluate and the second eluate, and combining the crystallized products.

In one embodiment of the present disclosure, the first eluate and the second eluate are respectively dried and recrystallized, and the crystalline products are combined.

In one embodiment of the present disclosure, the respectively drying and recrystallizing the first eluate and the second eluate comprises: dissolving the dried product of the first eluate and the dried product of the second eluate in acetone:water=1:1 to 3:1, and recrystallizing respectively.

It should be noted that the ratio between acetone and water is a volume ratio, and in the embodiments, the ratio between acetone and water is a volume ratio.

In one embodiment of the present disclosure, the respectively drying and recrystallizing the first eluate and the second eluate comprises: dissolving the dried product of the first eluate and the dried product of the second eluate in acetone:water=1:1 or 3:1, and recrystallizing respectively.

In one embodiment of the present disclosure, the respectively recrystallizing comprises: recrystallizing at 1° C. to 5° C.

In one embodiment of the present disclosure, recrystallization can be carried out at 4° C., 1° C. or 5° C.

In one embodiment of the present disclosure, the dissolution treatment in the step (1) is an ultrasonic treatment, and the treatment time is 30 to 60 minutes.

In one embodiment of the present disclosure, the dissolution treatment in the step (1) is an ultrasonic treatment, and the treatment time is 30 to 45 minutes.

In one embodiment of the present disclosure, the dissolution treatment in the step (1) is an ultrasonic treatment, and the treatment time is 45 to 60 minutes.

In one embodiment of the present disclosure, the dissolution treatment in the step (1) is an ultrasonic treatment, and the treatment time is 30 minutes, 45 minutes or 60 minutes.

In one embodiment of the present disclosure, the alkali solution is an aqueous Na2CO3 solution having a mass fraction of 0.1 to 0.4%.

The mass fraction is expressed in a % which means a g solute per 100 ml of the solvent.

In one embodiment of the present disclosure, the first alkali solution is an aqueous Na2CO3 solution having a mass fraction of 0.1 to 0.3%.

In one embodiment of the present disclosure, the alkali solution is an aqueous Na2CO3 solution having a mass fraction of 0.1 to 0.2%.

In one embodiment of the present disclosure, the alkali solution is an aqueous Na2CO3 solution having a mass fraction of 0.2 to 0.4%.

In one embodiment of the present disclosure, the alkali solution is an aqueous Na2CO3 solution having a mass fraction of 0.2 to 0.3%.

In one embodiment of the present disclosure, the alkali solution is an aqueous Na2CO3 solution having a mass fraction of 0.1%, 0.2%, 0.3% or 0.4%.

In one embodiment of the present disclosure, the step (1) comprises mixing each gram of Ginkgo biloba extract powder with 20 to 50 ml of the first alkali solution.

In one embodiment of the present disclosure, the step (1) comprises mixing each gram of Ginkgo biloba extract powder with 20 to 30 ml of the first alkali solution.

In one embodiment of the present disclosure, the step (1) comprises mixing each gram of Ginkgo biloba extract powder with 20 to 25 ml of the first alkali solution.

In one embodiment of the present disclosure, the step (1) comprises mixing each gram of Ginkgo biloba extract powder with 25 to 50 ml of the first alkali solution.

In one embodiment of the present disclosure, the step (1) comprises mixing each gram of Ginkgo biloba extract powder with 25 to 30 ml of the first alkali solution.

In one embodiment of the present disclosure, the step (1) comprises mixing each gram of Ginkgo biloba extract powder with 30 to 50 ml of the first alkali solution.

According to a second aspect of the present disclosure, a total ginkgolide prepared by the preparation method according to the first aspect is provided.

The present disclosure has the following beneficial effects:

The preparation method of total ginkgolide provided in the present disclosure can improve the purity of total ginkgolide, and the purity of the prepared total ginkgolide is ≥80%, which has good clinical application prospect; the preparation process does not require complicated multi-step recrystallization step; and the total ginkgolide yield is high.

### BRIEF DESCRIPTION OF THE DRAWINGS

FIG. 1 shows the precipitate results obtained in the step 12 of Example 1, the precipitate results are detected by high performance liquid chromatography-evaporative light scattering detector (HPLC-ELSD).

FIG. 2 shows the mixed standard of ginkgolide A, ginkgolide B, ginkgolide C and bilobalide detected by HPLC-ELSD.

FIG. 3 shows the results of component GJ-1 obtained in the step 22 of Example 1 detected by HPLC-ELSD.

FIG. 4 shows the results of component GJ-2 obtained in the step 22 of Example 1 detected by HPLC-ELSD.

### DETAILED DESCRIPTION OF THE PREFERRED EMBODIMENTS

Before the embodiments according to the present disclosure are further described, it should be understood that the protection scope of the present disclosure is not limited to the specific embodiments described below. It should also be understood that the term in the examples according to the present disclosure is used to describe the particular implementation, and is not intended to limit the protection scope of the present disclosure. In the specification and claims according to the present disclosure, unless otherwise stated specifically, the singular forms “a”, “an”, and “the” comprise the plural forms.

When the numerical ranges are given by the examples, it should be understood that the two endpoints of each numerical range and any numerical value between the two endpoints can be selected, unless otherwise stated herein. Unless otherwise defined, all technical and scientific terms used in the present disclosure have the same meaning as commonly understood by those skilled in the art. In addition to the specific methods, devices and materials, any methods, devices, and materials of the prior art that are similar or equivalent to the methods, devices, and materials described in the examples according to the present disclosure can also be used to implement the present disclosure in accordance with the prior art known by those skilled in the art and the description of the present disclosure.

### Example 1

1. Separating Total Ginkgolide

11. Ginkgo biloba extract powder (GBE50) with weight of 5 g was slowly added into 100 ml of aqueous Na2CO3 solution having a mass fraction of 0.3% (pH=9-10).

12. Ultrasonic dissolution was carried out for 60 minutes. Precipitate was obtained by high speed centrifugation at 10,000 r/min, and the precipitate was dried.

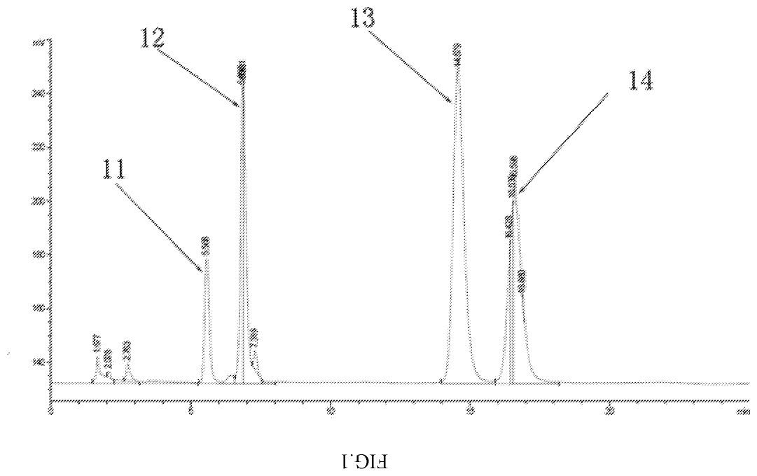

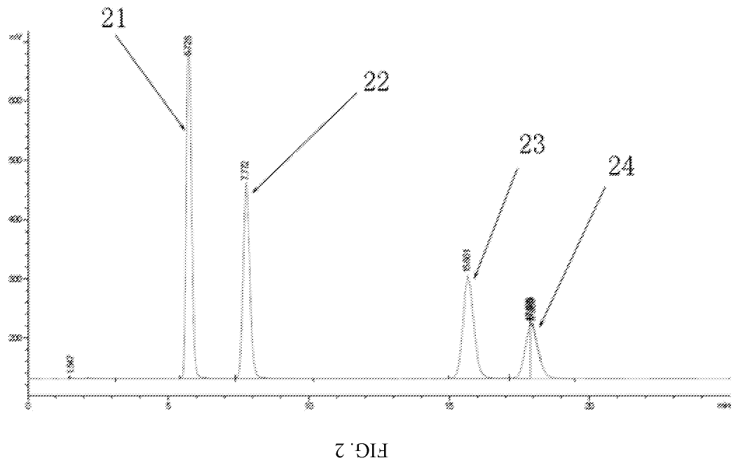

13. The precipitate obtained in the step 12 was detected by high performance liquid chromatography-evaporative light scattering detector (HPLC-ELSD). The results were shown in FIG. 1. Lines 11, 12, 13, and 14 in FIG. 1 indicate bilobalide, ginkgolide C, ginkgolide A, and ginkgolide B, respectively. Compared with FIG. 2 which shows the HPLC-ELSD test results for the mixed standard of ginkgolide A, ginkgolide B, ginkgolide C and bilobalide (lines 21, 22, 23, 24 in FIG. 2 indicate bilobalide, ginkgolide C, ginkgolide A, and ginkgolide B, respectively), it can be seen that the precipitate obtained in the step 12 includes ginkgolide A, ginkgolide B, ginkgolide C and bilobalide, and has little interfering impurities.

2. Enriching Total Ginkgolide

21. A silica gel column was prepared. 200-mesh silica gel was pretreated with a mixture of chloroform and methanol, chloroform:methanol=40:1. The column was packed by a wetting method, in which the ratio of diameter to height was 1:7, and the adsorption ratio was 1:40.

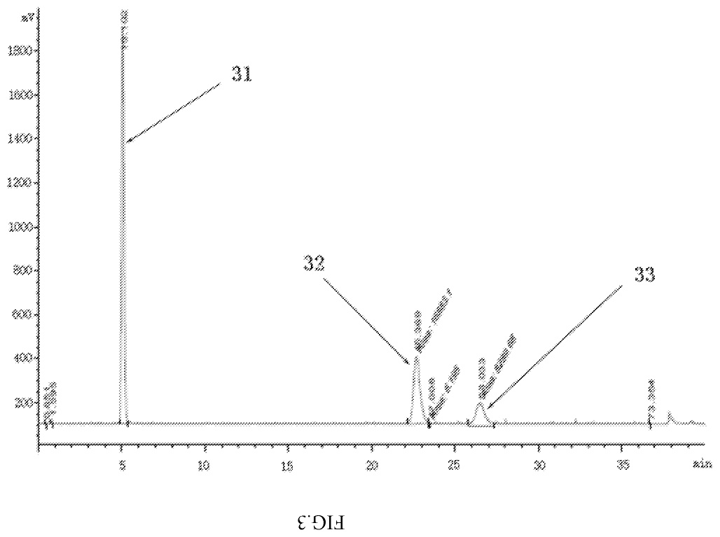

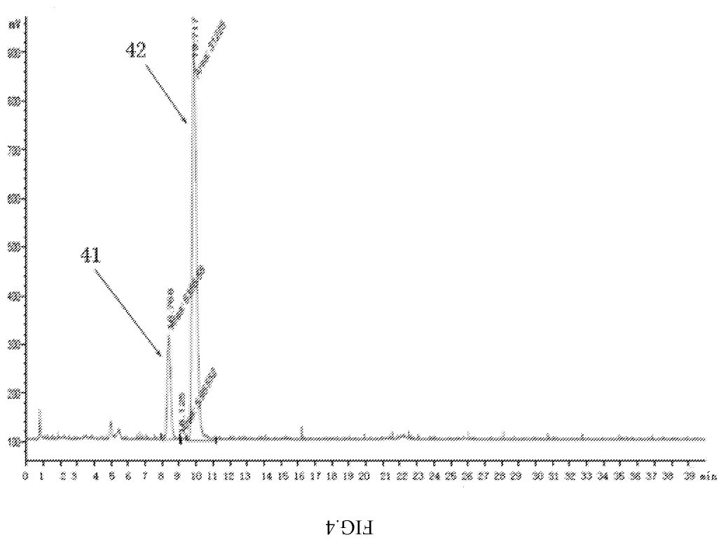

22. The precipitate obtained in the step 12 was dissolved in a mixture of chloroform and methanol, dichloromethane:methanol=1:1. The silica gel column prepared in the step 21 was loaded and washed with 5 column volumes of the first eluate and the second eluate in sequence. The first eluate was chloroform:methanol=40:1. The second eluate was chloroform:methanol=35:1. At the time of elution, the mixed standard of ginkgolide A, B, C, and bilobalide was used as a control, and compared eluted fractions with the control through thin layer chromatography, and the eluate containing the lactone component was combined. The component GJ-1 was obtained by eluting the first eluate, and component GJ-2 was obtained by eluting the second eluate. The component GJ-1 was detected by HPLC-ELSD, the content of ginkgolide in the component GJ-1 was 71.5%, and the results were shown in FIG. 3 (lines 31, 32, and 33 in FIG. 3 indicate bilobalide, ginkgolide A, and ginkgolide B, respectively). The component GJ-2 included the ginkgolide C which is not included in the component GJ-1, and the results were shown in FIG. 4 (lines 41 and 42 in FIG. 4 indicate bilobalide and ginkgolide C, respectively). A solid GJ-1 and a solid GJ-2 were obtained by drying the component GJ-1 and the component GJ-2, respectively.

23. Solid GJ-1 and solid GJ-2 were respectively dissolved with acetone solution with acetone:water=1:1, and recrystallized in a refrigerator at 4° C. The crystals were combined, and mixed crystals were detected by HPLC-ELSD, the content of ginkgolide was >90%, and the transfer rate of ginkgolide was 52%.

The total ginkgolide prepared in this example has a purity of ≥80%, and has good clinical application prospects; the preparation process does not require a complicated multi-step recrystallization step; and the total ginkgolide yield is high.

### Example 2

In this example, the 200-mesh silica gel in the step 21 was replaced with 300-mesh silica gel, and the other operations were the same as in Example 1. The obtained total ginkgolide had a purity of 88%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 3

In this example, the 200-mesh silica gel in the step 21 was replaced with 100-mesh silica gel, and the other operations were the same as in Example 1. The obtained total ginkgolide had a purity of 88%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 4

In this example, the ultrasonic treatment time in the step 12 was 45 minutes, and the other operations were the same as in Example 1. The obtained total ginkgolide had a purity of 92%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 5

In this example, the ultrasonic treatment time in the step 12 was 30 minutes, and the other operations were the same as in Example 1. The obtained total ginkgolide had a purity of 90%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 6

In this example, the step 11 included taking 1 g of GBE50 powder and adding 30 ml of aqueous Na2CO3 solution having a mass fraction of 0.3%, and the other operations were the same as in Example 1. The obtained total ginkgolide had a purity of 87%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 7

In this example, the step 11 included taking 10 g of GBE50 powder and adding 250 ml of aqueous Na2CO3 solution having a mass fraction of 0.3%, and the other operations were the same as in Example 1. The obtained total ginkgolide had a purity of 90%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 8

In this example, the step 11 included taking 2 g of GBE50 powder and adding 100 ml of aqueous Na2CO3 solution having a mass fraction of 0.3%, and the other operations were the same as in Example 1. The obtained total ginkgolide had a purity of 86%, and the transfer rate of ginkgolide was more than 50%.

The total ginkgolide prepared in this example had a purity of ≥80%, and had good clinical application prospects; and the preparation process did not require a complicated multi-step recrystallization step; the total ginkgolide yield was high.

### Example 9

In this example, the step 11 included taking 7 g of GBE50 powder and adding 300 ml of aqueous Na2CO3 solution having a mass fraction of 0.3%, and the other operations were the same as in Example 1. The obtained total ginkgolide had a purity of 87%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 10

In this example, the step 11 included taking 1 g of GBE50 powder and adding 50 ml of aqueous Na2CO3 solution having a mass fraction of 0.3%, and the other operations were the same as in Example 1. The obtained total ginkgolide had a purity of 90%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 11

In this example, the step 11 included taking 10 g of GBE50 powder and adding 200 ml of aqueous Na2CO3 solution having a mass fraction of 0.3%, and the other operations were the same as in Example 1. The obtained total ginkgolide had a purity of 91%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 12

In this example, the aqueous Na2CO3 solution having a mass fraction of 0.3% in the step 11 was replaced with the aqueous Na2CO3 solution having a mass fraction of 0.2%, and the other operations were the same as in Example 1. The obtained total ginkgolide had a purity of 92%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 13

In this example, the aqueous Na2CO3 solution having a mass fraction of 0.3% in the step 11 was replaced with the aqueous Na2CO3 solution having a mass fraction of 0.1%, and the other operations were the same as in Example 1. The obtained total ginkgolide had a purity of 93%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 14

In this example, the aqueous Na2CO3 solution having a mass fraction of 0.3% in the step 11 was replaced with the aqueous Na2CO3 solution having a mass fraction of 0.4%, and the other operations were the same as in Example 1. The obtained total ginkgolide had a purity of 91%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 15

In this example, the step 11 included taking 100 g of GBE50 powder and adding 2000 ml of aqueous Na2CO3 solution having a mass fraction of 0.3%, and the other operations were the same as in Example 1. The obtained total ginkgolide had a purity of 90%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 16

In this example, the recrystallization temperature in the step 23 was changed to 1° C., and the other operations were the same as in Example 1. The obtained total ginkgolide had a purity of 85%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 17

In this example, the recrystallization temperature in the step 23 was changed to 5° C., and the other operations were the same as in Example 1. The obtained total ginkgolide had a purity of 86%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 18

In this example, the recrystallization temperature in the step 23 was changed to 3° C., and the other operations were the same as in Example 1. The obtained total ginkgolide had a purity of 89%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 19

In this example, 300-mesh silica gel was pretreated with mixture of chloroform:methanol=40:1 in the step 21, and the other operations were the same as in Example 1. The obtained total ginkgolide had a purity of 90%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 20

In this example, 300-mesh silica gel was pretreated with mixture of chloroform:methanol=40:1 in the step 21, and the other operations were the same as in Example 1. The obtained total ginkgolide had a purity of 91%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 21

In this example, 250-mesh silica gel was pretreated with mixture of chloroform:methanol=10:1 in the step 21, and the other operations were the same as in Example 1. The obtained total ginkgolide had a purity of 89%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 22

In this example, 250-mesh silica gel was pretreated with mixture of chloroform:methanol=30:1 in the step 21, and the other operations were the same as in Example 1. The obtained total ginkgolide had a purity of 86%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 23

In this example, 250-mesh silica gel was pretreated with mixture of chloroform:methanol=50:1 in the step 21, and the other operations were the same as in Example 1. The obtained total ginkgolide had a purity of 89%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 24

In this example, 250-mesh silica gel was pretreated with mixture of chloroform:methanol=70:1 in the step 21, and the other operations were the same as in Example 1. The obtained total ginkgolide had a purity of 87%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 25

In this example, 250-mesh silica gel was pretreated with mixture of chloroform:methanol=100:1 in the step 21, and the other operations were the same as in Example 1. The obtained total ginkgolide had a purity of 89%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 26

In this example, the diameter to height ratio in the step 21 was 1:4, and the other operations were the same as in Example 1. The obtained total ginkgolide had a purity of 85%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 27

In this example, the diameter to height ratio in the step 21 was 1:6, and the other operations were the same as in Example 1. The obtained total ginkgolide had a purity of 88%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 28

In this example, the diameter to height ratio in step 21 was 1:8, and the other operations were the same as in Example 1. The obtained total ginkgolide had a purity of 86%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 29

In this example, the diameter to height ratio in the step 21 was 1:10, and the other operations were the same as in Example 1. The obtained total ginkgolide had a purity of 83%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 30

In this example, the precipitate obtained in the step 12 was dissolved with mixture of dichloromethane:methanol=2:1 in the step 22, and the other operations were the same as in Example 1. The obtained total ginkgolide had a purity of 88%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 31

In this example, the precipitate obtained in the step 12 was dissolved with mixture of dichloromethane:methanol=3:1 in the step 22, and the other operations were the same as in Example 1. The obtained total ginkgolide had a purity of 86%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 32

In this example, the first eluate in the step 22 was chloroform:methanol=42:1, and the other operations were the same as in Example 1. The obtained total ginkgolide had a purity of 86%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 33

In this example, the first eluate in the step 22 was chloroform:methanol=38:1, and the other operations were the same as in Example 1. The obtained total ginkgolide had a purity of 89%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 34

In this example, the second eluate in the step 22 was chloroform:methanol=33:1, and the other operations were the same as in Example 1. The obtained total ginkgolide had a purity of 88%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 35

In this example, the second eluate in the step 22 was chloroform:methanol=37:1, and the other operations were the same as in Example 1. The obtained total ginkgolide had a purity of 87%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 36

In this example, the dissolution was carried out with acetone:water=2:1 in the step 23, and the other operations were the same as in Example 1. The obtained total ginkgolide had a purity of 87%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 37

In this example, the dissolution was carried out with acetone:water=3:1 in the step 23, and the other operations were the same as in Example 1. The obtained total ginkgolide had a purity of 85%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

### Example 38

In this example, step 11 included taking 1,000 g of GBE50 powder and adding 20,000 ml of aqueous Na2CO3 solution having a mass fraction of 0.3%, and the other operations were the same as in Example 1. The obtained total ginkgolide had a purity of 89%, and the transfer rate of ginkgolide was more than 50%.

The total ginkgolide prepared in this example had a purity of ≥80%, and had good clinical application prospects; the preparation process did not require a complicated multi-step recrystallization step; and the total ginkgolide yield was high.

Further, the present disclosure has the following beneficial effects:

1) The present disclosure can make the purity of Ginkgo flavonoid glycosides to be ≥80%; 2) the final transfer rate of total ginkgolide according to the present disclosure is more than 50%, which is ideal; 3) 1,000 g raw material is tried in the present disclosure, which has good repeatability and good industrialization prospects; 4) the present disclosure does not require a complicated multi-step recrystallization step, and saves a large amount of low boiling point, toxic and harmful reagents such as petroleum ether and ethyl acetate.

In summary, the present disclosure effectively overcomes various shortcomings in the prior art and has high utilization value in industry.

The above-described examples are merely illustrative of the principles of the present disclosure and the effects thereof, and are not intended to limit the present disclosure. Modifications or variations of the above-described examples may be made by those skilled in the art without departing from the spirit and scope of the present disclosure. Therefore, all equivalent modifications or changes made by those skilled in the art without departing from the spirit and scope of the present disclosure will be covered by the appended claims.

1. A method for preparing total ginkgolide, comprising the following steps:
(1) mixing a Ginkgo biloba extract powder with an alkali solution, obtaining a precipitate by dissolving and centrifuging;
(2) obtaining a total ginkgolide concentrate through purifying the precipitate by a silica gel column and recrystallizing.

2. The preparation method as in claim 1, wherein
the filler in the silica gel column is silica gel.

3. The preparation method as in claim 2, wherein
the filler in the silica gel column is pretreated with chloroform:methanol=(10:1) to (100:1).

4. The preparation method as in claim 2, wherein
the filler in the silica gel column has a mesh number of 100 to 300.

5. The preparation method as in claim 1, wherein
the silica gel column has a diameter to height ratio of 1:4 to 1:10.

6. The preparation method as in claim 1, wherein
the absorption ratio of the silica gel column is 1:30 to 1:50.

7. The preparation method as in claim 1, wherein
step (2) comprises: dissolving the precipitate in dichloromethane:methanol=(1:1) to (3:1), and loading the silica gel column.

8. The preparation method as in claim 7, wherein
after loading the silica gel column, a first elution is carried out with dichloromethane:methanol=(42:1) to (38:1) to obtain a first eluate.

9. The preparation method as in claim 8, wherein
after the first elution, a second elution is carried out with dichloromethane:methanol=(37 to 33):1 to obtain a second eluate.

10. The preparation method as in claim 9, wherein
the first eluate and the second eluate are respectively dried and recrystallized, and the crystalline products are combined.

11. The preparation method as in claim 10, wherein respectively drying and recrystallizing the first eluate and the second eluate comprises:
dissolving the dried product of the first eluate and the dried product of the second eluate in solution of acetone:water=(1:1) to (3:1), and recrystallizing respectively.

12. The preparation method as in claim 11, wherein the respectively recrystallizing comprises:
recrystallizing at 1 to 5° C.

13. The preparation method as in claim 1, wherein
the dissolution treatment in the step (1) is an ultrasonic treatment, and the ultrasonic treatment time is 30 to 60 minutes.

14. The preparation method as in claim 1, wherein
the alkali solution is an aqueous Na₂CO₃ solution having a mass fraction of 0.1% to 0.4%.

15. The preparation method as in claim 1, wherein
the step (1) comprises mixing each gram of Ginkgo biloba extract powder with 20 ml to 50 ml of the alkali solution.

16. Total ginkgolide prepared by a preparation method, the method comprises the following steps:
(1) mixing a Ginkgo biloba extract powder with an alkali solution, obtaining a precipitate by dissolving and centrifuging;
(2) obtaining a total ginkgolide concentrate through purifying the precipitate by a silica gel column and recrystallizing.
